(19) United States
(12) Patent Application Publication
(10) Pub. No.: US 2015/0374954 A1
(43) Pub. Date: Dec. 31, 2015

(54) METHOD OF PROVIDING POST OPERATIVE EPIDURAL ANALGESIA IN SPINAL SURGERY

(71) Applicant: Edgar Newman WEAVER, JR., Roanoke, VA (US)

(72) Inventor: Edgar Newman WEAVER, JR., Roanoke, VA (US)

(21) Appl. No.: 14/320,626

(22) Filed: Jun. 30, 2014

(51) Int. Cl. A61M 25/01

(52) U.S. Cl. CPC A61M 25/0113; A61M 25/0108; A61M 2210/1003

(57) ABSTRACT

A method of placing spinal epidural catheters prior, during or after spinal surgery is provided. The method may include an epidural catheter tunneled externally down an epidular space above the operative site so as to continuously infuse analgesic solution through the catheter into a predetermined site.

### BACKGROUND OF THE INVENTION

The present invention relates to surgical methods and, more particularly, to a method for providing post operative epidural analgesia in a spinal surgery.

Typically after surgery a patient experiences severe post-operative pain. In response, to control such post-operative pain, placement of epidural catheters is usually done percutaneously—i.e., via a spinal epidural catheter. However, such placement is not feasible for most spinal cases as it would encroach on the operative field if placed before surgery, or would have to be placed through the operative field if placed after surgery.

As can be seen, there is a need for improved methods of placing spinal epidural catheters before, during or after spinal surgery.

### SUMMARY OF THE INVENTION

In one aspect of the present invention, method of an epidural surgical procedure comprises: providing a steerable catheter with a distal end and a proximal end; tunneling the distal end downward through an incision and through an epidural space of a patient, wherein the incision is placed above an operative site; and passing an analgesic solution through the proximal end and into a lumen of the steerable catheter disposed within a predetermined epidural space.

In another aspect of the present invention, a method of an epidural surgical procedure, comprises: providing a steerable catheter with a distal end and a proximal end; marking the catheter with markings; connecting a flattened bullet-head tip to the distal end; providing a stylet; providing a forceps holder for grasping the catheter; tunneling the distal end downward through an incision placed two vertebral levels up from an operative site and through an epidural space of a patient; grasping the catheter at a predetermined site between the incision and the operative site so as to steer the catheter to the predetermined epidural space; looping the catheter cephalad so that the distal end may be in the predetermined epidural space; and passing an analgesic solution through the proximal end and into a lumen of the steerable catheter disposed within a predetermined epidural space.

In another aspect of the present invention, a method of an epidural surgical procedure comprises: providing a long peel-away introducer; tunneling the long peel-away introducer downward through an incision and through an epidural space of a patient, wherein the incision is above an operative site; providing a steerable catheter with a distal end and a proximal end; tunneling the distal end downward through the long peel-away introducer and into the epidural space; and passing an analgesic solution through the proximal end and into a lumen of the steerable catheter disposed within a predetermined epidural space.

These and other features, aspects and advantages of the present invention will become better understood with reference to the following drawings, description and claims.

### BRIEF DESCRIPTION OF THE DRAWINGS

FIG. 1 is a flow chart of an exemplary embodiment of the present invention;

FIG. 2 is a schematic view of an exemplary embodiment of the present invention; and

FIG. 3 is a perspective and detailed view of an exemplary embodiment of a forceps holder and a stylet of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method of placing spinal epidural catheters before, during or after spinal surgery. The method may include an epidural catheter tunneled externally down an epidular space above the operative site so as to continuously infuse analgesic solution through the catheter into a predetermined site.

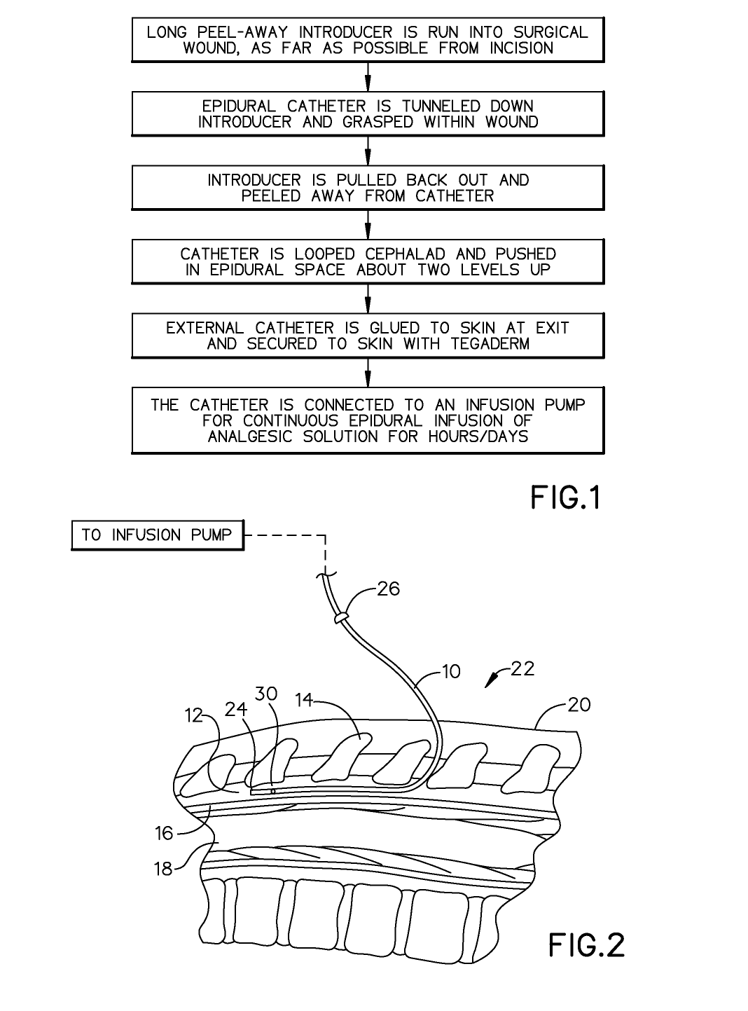

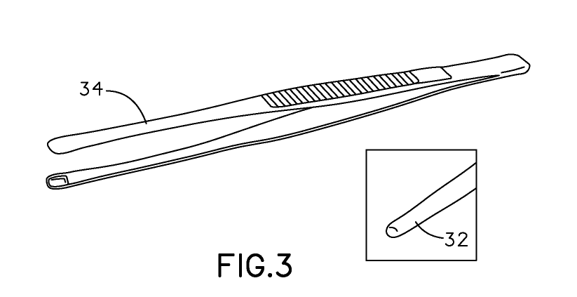

Referring to FIG. 1, the present invention may be a directly-visualized method of placing spinal epidural catheters before, during or after spinal surgery 100. The method 100 may include a catheter 10 having a distal end 24 and a proximal end. The proximal end may extend so as to be secured over the shoulder of the patient with a medical fastener and without need for attaching to the back of the patient, which can be uncomfortable. The distal end 24 may include a tip. The tip may include a flattened bullet-head for aiding advancement, yet adapted to be withdrawn percutaneously. The catheter may include a stylet 32 adapted for shaping of catheter distal end 24 and/or tip and provides added stiffness to assist in adequate advancement.

The method 100 may include introducing a long peel-away introducer 26 into the patient above an operative site 22, in step 110. The long peel-away introducer 26 may be adapted for placement of the catheter 10 in the surgery site via the skin from some distance away from any bandaging present so that the bandaging may not cover exiting catheter and so may be changed without disrupting the catheter 10. In certain embodiments, the operative site 22 may be prepared to expose an epidural space 12 for easy introduction of the introducer 26 and/or catheter 10. In certain embodiment, the introduction of the long peel-away introducer 26 may be made as far as possible from an incision above the operative site 22. Preferably, the introducer 26/catheter 10 may be placed two vertebral levels up from the operative site 22. It has been empirically determined that the two vertebral levels may be necessary for optimal pain control. The introducer 26 may be visually directed down the spinal cord 18 between a dural sac 16 and a spinous process 14. In certain embodiments, catheter 10 and/or distal end 24 may be marked by at least one marking 30 detectable with x-ray or by means of other imagining techniques so as to verify the placement relative to (internal) portions of the operative site 22.

Then in step 120, an epidural catheter 10 may be tunneled externally down the introducer 26. In certain embodiments, the catheter 10 may be tunneled down by use of a forceps catheter holder 34. In certain embodiments, the catheter 10 may be grasped within the operative site 22, wound site and/or other predetermined site.

The introducer 26 may be pulled back out and peeled away from the catheter 10, in step 130. Then the catheter 10 may be looped towards the head, anterior part and/or cephalad so that the distal end 24 may be in the epidural space 12 above the operative site 22 about two levels up, in step 140. The external portion of the catheter 10 may be removably attached to a portion of the patient's skin 20 at exit and secured to the skin 20 by a medical fastener, in step 150. It should be understood that the medical fastener may be any medical fastener known in the art for fastening or removably securing a medical device to the skin 20 of a patient, such as tegaderm or the like. In step 160, the proximal end of the catheter 10 may be connected to an infusion pump, as illustrated in FIG. 2, so as to continuously infuse analgesic solution through the catheter 10 to the epidural space 12 above the operative site 22.

It should be understood, of course, that the foregoing relates to exemplary embodiments of the invention and that modifications may be made without departing from the spirit and scope of the invention as set forth in the following claims.

What is claimed is:

1. A method of an epidural surgical procedure comprising:
providing a steerable catheter with a distal end and a proximal end;
tunneling the distal end downward through an incision and through an epidural space of a patient, wherein the incision is placed above an operative site; and
passing an analgesic solution through the proximal end and into a lumen of the steerable catheter disposed within a predetermined epidural space.

2. The method of claim 2, wherein the incision is placed two vertebral levels up from the operative site.

3. The method of claim 1, further including connecting a flattened bullet-head tip to the distal end.

4. The method of claim 1, further including providing a stylet.

5. The method of claim 1, further including grasping the catheter at a predetermined site between the incision and the operative site so as to steer the catheter to the predetermined epidural space.

6. The method of claim 5, further including providing a forceps holder for grasping the catheter.

7. The method of claim 1, further including looping the catheter cephalad so that the distal end may be in the predetermined epidural space.

8. The method of claim 1, wherein the proximal end extends over the shoulder of the patient.

9. The method of claim 1, further including marking the catheter with markings.

10. A method of an epidural surgical procedure comprising:
providing a long peel-away introducer;
tunneling the long peel-away introducer downward through an incision and through an epidural space of a patient, wherein the incision is above an operative site;
providing a steerable catheter with a distal end and a proximal end;
tunneling the distal end downward through the long peel-away introducer and into the epidural space; and
passing an analgesic solution through the proximal end and into a lumen of the steerable catheter disposed within a predetermined epidural space.

11. The method of claim 10, further including peeling away the long peel-away introducer.

12. The method of claim 10, wherein the incision is placed two vertebral levels up from the operative site.

13. The method of claim 10, further including connecting a flattened bullet-head tip to the distal end.

14. The method of claim 10, further including providing a stylet.

15. The method of claim 10, further including grasping the catheter at a predetermined site between the incision and the operative site so as to steer the catheter to the predetermined epidural space.

16. The method of claim 15, further including providing a forceps holder for grasping the catheter.

17. The method of claim 10, further including looping the catheter cephalad so that the distal end may be in the predetermined epidural space.

18. The method of claim 10, wherein the proximal end extends over the shoulder of the patient.

19. The method of claim 10, further including marking the catheter with markings.

20. A method of an epidural surgical procedure, comprising:
providing a steerable catheter with a distal end and a proximal end;
marking the catheter with markings;
connecting a flattened bullet-head tip to the distal end;
providing a stylet;
providing a forceps holder for grasping the catheter;
tunneling the distal end downward through an incision placed two vertebral levels up from an operative site and through an epidural space of a patient;
grasping the catheter at a predetermined site between the incision and the operative site so as to steer the catheter to the predetermined epidural space;
looping the catheter cephalad so that the distal end may be in the predetermined epidural space; and
passing an analgesic solution through the proximal end and into a lumen of the steerable catheter disposed within a predetermined epidural space.
